(19) United States
(12) Patent Application Publication
(10) Pub. No.: US 2025/0195285 A1
(43) Pub. Date: Jun. 19, 2025

(54) URINE-BLOCKING AND ABSORBING PAD FOR A CHILD

(71) Applicant: Amy Smith, Beltsville, MD (US)

(72) Inventor: Amy Smith, Beltsville, MD (US)

(21) Appl. No.: 18/545,703

(22) Filed: Dec. 19, 2023

(51) Int. Cl. A61F 13/47; A61L 15/24

(52) U.S. Cl. CPC A61F 13/4702; A61L 15/24

(57) ABSTRACT

A urine-blocking and absorbing pad to cover a pelvic area of a child during a diaper-change, the urine-blocking and absorbing pad including a waterproof layer to prevent urine from exiting the urine-blocking and absorbing pad, a fluff layer to contact the pelvic area of the child, and an absorbing layer disposed between the waterproof layer and the fluff layer to absorb the urine.

### BACKGROUND

### 1. Field

The present general inventive concept relates generally to a pad, and particularly, to urine-blocking and absorbing pad for a child and/or baby.

### 2. Description of the Related Art

For parents and caretakers, changing diapers can often be a race against time, especially when changing male baby diapers, as often there is a likelihood of urine release from the baby during the diaper-changing process. Whether caused by temperature changes or the randomness of bodily functions, once this unexpected stream of urine begins to spray from the child, the parent tends to get wet, along with the child and the new clean diaper, and clean-up of the area and/or diaper replacement is required. This extends the time to perform the diaper-changing task and creates waste for quickly and accidentally soiled diapers.

Therefore, there is a need for a modern accessory designed to prevent any and all unexpected and messy surprises during diaper changes, allowing for an efficient and improved changing experience.

There is also a need to provide users with a protective absorbent shield while changing diapers.

### SUMMARY

The present general inventive concept provides a urine-blocking and absorbing pad.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing urine-blocking and absorbing pad to cover a pelvic area of a child during a diaper-change, the urine-blocking and absorbing pad including a waterproof layer to prevent urine from exiting the urine-blocking and absorbing pad, a fluff layer to contact the pelvic area of the child, and an absorbing layer disposed between the waterproof layer and the fluff layer to absorb the urine.

The urine-blocking and absorbing pad may have an overall concave shape to conform to the pelvic area of the child.

The absorbing layer may include sodium polyacrylate.

The fluff layer may include at least one of a hydrophilic fibrous nonwoven fabric, a composite sheet composed of a hydrophobic fibrous nonwoven fabric, a breathable liquid-impervious plastic film laminated together, a water repellent finished hydrophobic fibrous nonwoven fabric, a mixture of particulate and/or fibrous superabsorbent polymers, fluff pulp fibers, a mixture of particulate and/or fibrous superabsorbent polymers, thermoplastic synthetic resin fibers, cotton, and tissue paper.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a top perspective view of a urine-blocking and absorbing pad, according to an exemplary embodiment of the present general inventive concept;

FIG. 1B illustrates a bottom perspective view of the urine-blocking and absorbing pad of FIG. 1A, according to an exemplary embodiment of the present general inventive concept;

FIG. 1C illustrates a side cross-sectional view of the urine-blocking and absorbing pad of FIGS. 1A and 1B, according to an exemplary embodiment of the present general inventive concept; and

FIG. 2 illustrates a top perspective view of another urine-blocking and absorbing pad, according to another exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### List of Components

Urine-Blocking and Absorbing Pad 100

Waterproof Layer 110

Absorbing Layer 120

Fluff Layer 130

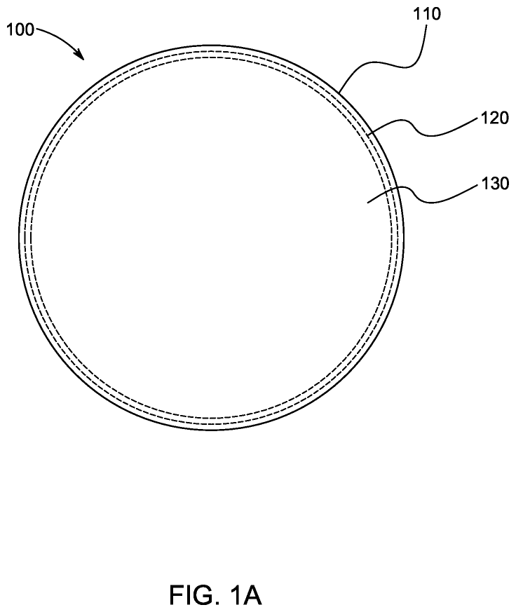

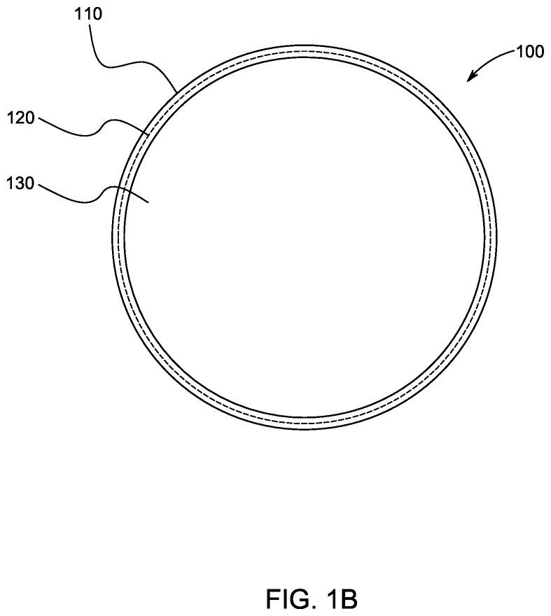

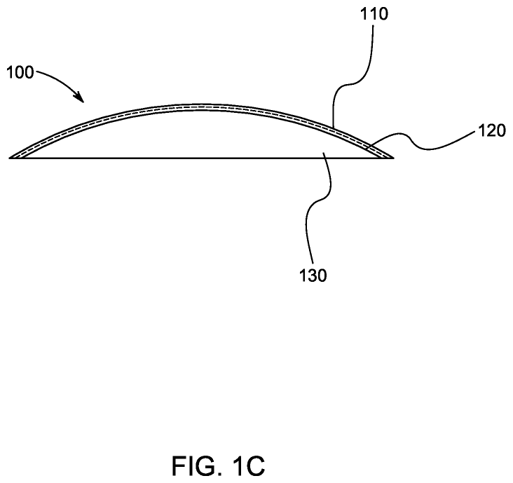

FIG. 1A illustrates a top perspective view of a urine-blocking and absorbing pad 100, according to an exemplary embodiment of the present general inventive concept.

FIG. 1B illustrates a bottom perspective view of the urine-blocking and absorbing pad 100 of FIG. 1A, according to an exemplary embodiment of the present general inventive concept; and

FIG. 1C illustrates a side cross-sectional view of the urine-blocking and absorbing pad 100 of FIGS. 1A and 1B, according to an exemplary embodiment of the present general inventive concept.

Referring to FIGS. 1A through 1C, the urine-blocking and absorbing pad 100 has an overall round shape, but is not limited thereto.

The urine-blocking and absorbing pad 100 may be 2 to 4 inches in diameter, but is not limited thereto.

Materials that are included within the urine-blocking and absorbing pad 100 may be flexible to allow the urine-blocking and absorbing pad 100 to conform to a child's body.

Specifically, the urine-blocking and absorbing pad 100 may be placed over a child's pelvic area during a diaper-change, such that a penis or a vagina of a child is covered. If the child begins to urinate while the diaper is being changed, the urine-blocking and absorbing pad 100 catches and absorbs the urine, such that a parent and/or a childcare giver does not get sprayed with the urine.

Furthermore, all of the materials within the entire urine-blocking and absorbing pad 100 may be bio-degradable, such that the urine-blocking and absorbing pad 100 is disposable.

Referring to FIGS. 1A through 1C, the urine-blocking and absorbing pad 100 may include a waterproof layer 110, an absorbing layer 120, and a fluff layer 130, but is not limited thereto.

Referring to FIGS. 1A through 1C, the waterproof layer 110 may be a topmost later of the urine-blocking and absorbing pad 100.

The waterproof layer 110 may be created from rubber, plastic, or any other type of material that is waterproof, such that a liquid cannot penetrate the waterproof layer 110.

The waterproof layer 110 provides an outer-shell for the urine-blocking and absorbing pad 100, and may have a concave shape that conforms to a child's pelvic area.

Referring to FIGS. 1A through 1C, the absorbing layer 120 is a layer of the urine-blocking and absorbing pad 100 that is disposed between the waterproof layer 110 and the fluff layer 130.

The absorbing layer 120 may be materials created from solids, liquids, gels, powder, and/or crystals, such that the absorbing later 120 is able to absorb liquid, such as urine.

For example, the absorbing layer 120 may be created from sodium polyacrylate, which is well known to have absorptive properties.

Referring to FIGS. 1A through 1C, the fluff layer 130 is the actual portion of the urine-blocking and absorbing pad 100 that contacts the skin of the child, and therefore, may be soft as to avoid irritating the skin of the child.

The fluff layer 130 may be created from any combination of soft materials, including, but not limited to, a hydrophilic fibrous nonwoven fabric, a composite sheet composed of a hydrophobic fibrous nonwoven fabric, a breathable liquid-impervious plastic film laminated together, a water repellent finished hydrophobic fibrous nonwoven fabric, a mixture of particulate and/or fibrous superabsorbent polymers, fluff pulp fibers, a mixture of particulate and/or fibrous superabsorbent polymers, thermoplastic synthetic resin fibers, cotton, and tissue paper.

The fluff layer 130 may include any of the above-mentioned soft materials compressed into a relatively thin thickness, such that a concave shape is formed to correspond to the absorbing later 120 and the waterproof layer 110.

As such, when a child soils its diaper, a parent and or childcare provider may remove the diaper and immediately cover the child's pelvic area with urine-blocking and absorbing pad 100. If the child begins to unexpectedly urinate prior to a new diaper being applied to the child, the urine will first hit the fluff layer 130, then will be absorbed in the absorption layer 120, and then will be blocked by the waterproof layer 110. As such, the parent and/or childcare provider will not be hit with the unexpected stream of urine. Also, prior to the diaper being closed, the urine-blocking and absorbing pad 100 may be removed and reused if it is not soiled.

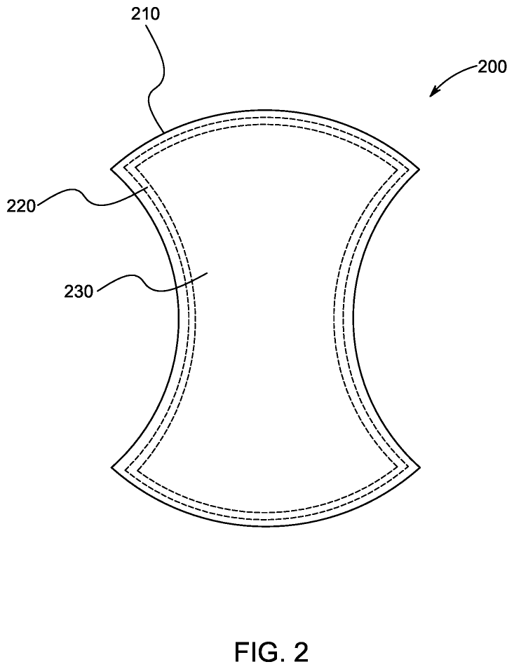

FIG. 2 illustrates a top perspective view of another urine-blocking and absorbing pad 200, according to another exemplary embodiment of the present general inventive concept.

Referring to FIG. 2, the urine-blocking and absorbing pad 200 may have a shape that conforms to a child's body, such as a shape that has concave side portions and convex upper and bottom portions, as illustrated in FIG. 2, but is not limited thereto, and may have any other shape that is more ergonomically-designed to conform to a child's body.

Materials that are included within the urine-blocking and absorbing pad 200 may be flexible to allow the urine-blocking and absorbing pad 200 to conform to a child's body.

Specifically, the urine-blocking and absorbing pad 200 may be placed over a child's pelvic area during a diaper-change, such that a penis or a vagina of a child is covered. If the child begins to urinate while the diaper is being changed, the urine-blocking and absorbing pad 200 catches and absorbs the urine, such that a parent and/or a childcare provider does not get sprayed with the urine.

Furthermore, all of the materials within the entire urine-blocking and absorbing pad 200 may be bio-degradable, such that the urine-blocking and absorbing pad 200 is disposable.

Referring to FIG. 2, the urine-blocking and absorbing pad 200 may include a waterproof layer 210, an absorbing layer 220, and a fluff layer 230, but is not limited thereto.

Referring to FIG. 2, the waterproof layer 210 may be a topmost later of the urine-blocking and absorbing pad 200.

The waterproof layer 210 may be created from rubber, plastic, or any other type of material that is waterproof, such that a liquid cannot penetrate the waterproof layer 210.

The waterproof layer 210 provides an outer-shell for the urine-blocking and absorbing pad 200, and may have a concave shape that conforms to a child's pelvic area.

Referring to FIG. 2, the absorbing layer 220 is a layer of the urine-blocking and absorbing pad 200 that is disposed between the waterproof layer 210 and the fluff layer 230.

The absorbing layer 220 may be materials created from solids, liquids, gels, powder, and/or crystals, such that the absorbing later 220 is able to absorb liquid, such as urine.

For example, the absorbing layer 220 may be created from sodium polyacrylate, which is well known to have absorptive properties.

Referring to FIG. 2, the fluff layer 230 is the actual portion of the urine-blocking and absorbing pad 200 that contacts the skin of the child, and therefore, may be soft as to avoid irritating the skin of the child.

The fluff layer 230 may be created from any combination of soft materials, including, but not limited to, a hydrophilic fibrous nonwoven fabric, a composite sheet composed of a hydrophobic fibrous nonwoven fabric, a breathable liquid-impervious plastic film laminated together, a water repellent finished hydrophobic fibrous nonwoven fabric, a mixture of particulate and/or fibrous superabsorbent polymers, fluff pulp fibers, a mixture of particulate and/or fibrous superabsorbent polymers, thermoplastic synthetic resin fibers, cotton, and tissue paper.

The fluff layer 230 may include any of the above-mentioned soft materials compressed into a relatively thin thickness, such that a concave shape is formed to correspond to the absorbing later 220 and the waterproof layer 210.

As such, when a child soils its diaper, a parent and or childcare provider may remove the diaper and immediately cover the child's pelvic area with urine-blocking and absorbing pad 200. If the child begins to unexpectedly urinate prior to a new diaper being applied to the child, the urine will first hit the fluff layer 230, then will be absorbed in the absorption layer 220, and then will be blocked by the waterproof layer 210. As such, the parent and/or childcare provider will not be hit with the unexpected stream of urine. Also, prior to the diaper being closed, the urine-blocking and absorbing pad 200 may be removed and reused if it is not soiled.

The present general inventive concept may include a urine-blocking and absorbing pad 100 to cover a pelvic area of a child during a diaper-change, the urine-blocking and absorbing pad 100 including a waterproof layer 110 to prevent urine from exiting the urine-blocking and absorbing pad 100, a fluff layer 130 to contact the pelvic area of the child, and an absorbing layer 120 disposed between the waterproof layer 110 and the fluff layer 130 to absorb the urine.

The urine-blocking and absorbing pad 100 may have an overall concave shape to conform to the pelvic area of the child.

The absorbing layer 120 may include sodium polyacrylate.

The fluff layer 130 may include at least one of a hydrophilic fibrous nonwoven fabric, a composite sheet composed of a hydrophobic fibrous nonwoven fabric, a breathable liquid-impervious plastic film laminated together, a water repellent finished hydrophobic fibrous nonwoven fabric, a mixture of particulate and/or fibrous superabsorbent polymers, fluff pulp fibers, a mixture of particulate and/or fibrous superabsorbent polymers, thermoplastic synthetic resin fibers, cotton, and tissue paper.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A urine-blocking and absorbing pad to cover a pelvic area of a child during a diaper-change, the urine-blocking and absorbing pad comprising:
a waterproof layer to prevent urine from exiting the urine-blocking and absorbing pad;
a fluff layer to contact the pelvic area of the child; and
an absorbing layer disposed between the waterproof layer and the fluff layer to absorb the urine.

2. The urine-blocking and absorbing pad of claim 1, wherein the urine-blocking and absorbing pad has an overall concave shape to conform to the pelvic area of the child.

3. The urine-blocking and absorbing pad of claim 1, wherein the absorbing layer comprises sodium polyacrylate.

4. The urine-blocking and absorbing pad of claim 1, wherein the fluff layer comprises at least one of a hydrophilic fibrous nonwoven fabric, a composite sheet composed of a hydrophobic fibrous nonwoven fabric, a breathable liquid-impervious plastic film laminated together, a water repellent finished hydrophobic fibrous nonwoven fabric, a mixture of particulate and/or fibrous superabsorbent polymers, fluff pulp fibers, a mixture of particulate and/or fibrous superabsorbent polymers, thermoplastic synthetic resin fibers, cotton, and tissue paper.
